(19) United States
(12) Patent Application Publication
(10) Pub. No.: US 2012/0132784 A1
(43) Pub. Date: May 31, 2012

(54) AMBULATORY INTRAVENOUS (IV) TRANSPORT AND DELIVERY DEVICE and METHOD of USE THEREOF

(76) Inventors: Delmar Sean Dukes, Jamison, PA (US); Avery Lauren Dukes, Jamison, PA (US)

(21) Appl. No.: 13/305,337

(22) Filed: Nov. 28, 2011

Related U.S. Application Data
(60) Provisional application No. 61/417,512, filed on Nov. 29, 2010.

(51) Int. Cl. A61M 5/00

(52) U.S. Cl. 248/690

(57) ABSTRACT

Provided are device, system, and method for storing and/or transporting a fluid-filled pouch for ambulatory intravenous (“IV”) transport and delivery of IV solutions to a fully mobile patient, wherein the IV fluid pouch is maintained and constantly infuses the patient from a constantly elevated position relative to the insertion point on the patient, while at the same time providing the patient with free, unrestricted range of motion without requiring any pump or electrical or mechanical connection to maintain IV infusion, in any location indoors or out, regardless of the terrain or patient's activity.

### REFERENCE TO RELATED APPLICATION

This application claims priority to U.S. Provisional Application No. 61/417,512, filed Nov. 29, 2011, which is herein incorporated by reference in its entirety.

### FIELD OF THE INVENTION

The invention relates to a device, system, and method for ambulatory intravenous (“IV”) transport and delivery of IV solutions to a mobile patient.

### BACKGROUND

Intravenous delivery of fluids to a patient requires that pressure be exerted upon the fluids to be delivered such that the pressure exerted upon the fluid being forced into the body is greater than the pressure exerted upon fluids presently in the body. This can be accomplished in a variety of ways, however most commonly, the fluid being infused into the patient is held in a reservoir, pouch, or bag located above the insertion point on the patient. In this way, gravity acts upon the fluid and overcomes the internal fluid pressures in the patient's body. However, in order to maintain this pressure imbalance, it is necessary to continuously suspend the IV pouch in an elevated position above the highest point on the body of the patient.

As a result, a variety of devices and systems have been developed to maintain pouches in a constantly elevated position above the patient. For example, some systems are stationary stands that allow the pouches to be suspended from them, while others are ambulatory devices that allow the user to suspend the fluid from a pole on wheels. But, of course, both of these types of system require a solid, flat surface on which to rest the stand, including one with wheels. Accordingly, a patient receiving an IV infusion is thus confined to areas that will support the IV pole, and the patient cannot even move from one flat location to another if stairs, rough ground or other features are involved.

Although systems have been developed that allow a patient to wear a fanny-pack type device that contains the IV fluid pouch, such systems require the use of an electrical or mechanical pump to infuse the fluids into the patient's body. Such systems cannot operate by gravity feed, and as a result, the patient is still limited by the range and weight of the pump systems, which cannot operate without a source of power. Accordingly, such systems offer alternatives to requiring the patient to be attached to a wheeled pole mounted system, but the patient is still restricted to limited mobility.

As a result, there has remained a need in the art until the present invention for a device and system that provides for ambulatory intravenous (“IV”) transport and delivery of IV solutions to a fully mobile patient, wherein the IV fluid pouch is maintained in a constantly elevated position relative to the insertion point on the patient, while at the same time providing the patient with free, unrestricted range of motion without requiring any pump or electrical or mechanical connection to maintain IV infusion in any location indoors or out.

### SUMMARY OF THE INVENTION

The present invention provides a device and/or system and methods for such use to facilitate medical support to an ambulatory patient in need of intravenous (“IV”) infusion, advantageously and expressly without requiring the mobility-limiting fixed or wheeled poles known in the art for holding such infusion pouches during gravity-fed delivery of the medical fluid to the individual. The device comprises at least two parts: a backpack-type exoframe (“exoskeleton”) of light-weight material, hollow tubing, comprising two vertical tubular support elements (“poles”) and operably attached horizontal supporting members between the two vertical supports, wherein the topmost end of at least one of the two vertical poles is open. An adjustable pole extends from the open end of the vertical tubing and a medical fluid pouch, specifically an intravenous fluid pouch, is suspended from the top of the exoskeleton frame. In such position, when in operable use as worn by a wearer W, the adjustable pole extends vertically, to a point above the head of the wearer W (placing the bottom of the pouch above the shoulder of the wearer). When the adjustable pole is in place, extending to be the uppermost pole of the exoskeleton, a medical fluid bag, pouch or container, as the terms are herein used interchangeably, is hung from the top of the vertical extension pole.

A pack when operably attached to the exoframe allows the user to carry the typical contents of backpacks: sleeping bag, camping equipment, search and rescue equipment, medical equipment or items for military operations and the like. In addition, the components of the invention, such as a retractable, telescoping adjustable pole, if removed from its operable position, and additional IV supplies, may be stowed or carried in the pack when the pack is present on the exoframe. However, the invention is operable so long as the exoframe is available, regardless of the presence of the pack, which can be of any construction known in the art for hollow, tubular exoframe supported backpack-type carriers.

In an exemplary embodiment, the present invention comprises at least one vertical adjustable support pole (the “adjustable IV pole”) that extends from the open vertical pole of the frame “exoskeleton” (also referred to herein as the “frame or “exoframe”) worn on a wearer W's back (“shoulder-borne”) or if there is a cross-piece, when the original top cross-piece of the backpack exoskeleton is removed. The final result in either embodiment is that the top of the vertical support on either and/or both sides of the exoframe are open to receive the adjustable IV pole of the present invention. The adjustable IV pole, sized to mate with the vertical pole of the exoskeleton, is selected having two ends: one end is an open end, while the opposing end of the pole provides a clamp, loop or hook in operably interchangeable form, for holding a medical IV infusion pouch in operable position.

The open end of the adjustable IV pole is preferably inserted into the opening at the top of the exoframe of the backpack, and held in place by friction or by spring or clip features or buttons to prevent the lower end of the adjustable pole from sliding out of position within the vertical pole of the frame of the backpack. Alternately, in one embodiment, the adjustable IV pole is larger than the vertical pole of the exoskeleton, and thus slips into place over the vertical pole, rather than being inserted into the vertical pole. In certain embodiments, the adjustable IV pole is always integrated into (stored within or deployed from) the exoskeleton of the backpack, meaning that it is deployed as needed. Otherwise, the backpack functions in accordance with known backpack designs and functions, including use for storage and/or transport of items carried therein.

When in operable position in the invention, the open end of the adjustable pole is placed into the open end of the vertical pole of the exoframe, so that the adjustable pole extends vertically above the head of the wearer W (placing the bottom of the pouch above the shoulder of the wearer), when the backpack is shoulder-borne. Thus, when a medical fluid IV pouch is in use, it is suspended from the clamp, loop or hook at the top of the affixed adjustable pole; which assumes that in a preferred embodiment, when in operation, the IV infusion is gravity fed. When used for example, for camping, search and rescue, medical equipment for military operations, the pole may be stored within the exoframe structure, and need not be deployed from the exoskeleton until it is needed.

Alternative embodiments and methods of use are also provided, wherein the cross-piece is removed and the exoframe has two vertical support posts, each of which is left open at the top. Two adjustable IV poles may be added, one into each opening of the exoframe of the backpack, wherein at least one of the two adjustable poles has a clamp, loop, or hook as described above, to operably support one or more medical IV fluid pouches.

Advantageously, when the present invention is assembled and the adjustable pole is extended and locked into position, there is provided a system and method for hanging at least one medical IV fluid pouch from the top of at least one disclosed adjustable IV pole, while giving the wearer W full range of motion. Once configured, the individual can walk, jog or run while the fluid is being delivered to the wearer patient, without being encumbered by fixed or wheeled support, or by electrical or mechanical pumps. In fact, the present invention includes no electrical or mechanical connection to any fixed apparatus. It has no foot, base or wheels tethering the wearer W, nor does it offer any tripping or knotting hazard.

In a useful alternative embodiment, wearer W wears the present invention and suspends the medical IV fluid from the device while the fluid is being infused into someone else adjacent to wearer W, thereby allowing the wearer of the device to carry, support or transport the individual being infused.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

For the purpose of illustration, there are depicted in the drawings certain non-limiting embodiments of the invention. However, the invention is non-limited to the precise arrangements and instrumentalities of the embodiments depicted in the drawings. Reference numbers are used consistently among the Figures.

FIG. 1 shows the front view of a backpack-type exoskeleton, as worn by wearer (“W”), with an ambulatory intravenous socket hanger system in an exemplary embodiment as depicted in a deployed state. The pack portion of the backpack is not shown to more easily view the exoskeleton. The IV bag is shown to provide orientation of the components of the invention for display purposes.

FIG. 2 shows a side view of the embodiment shown in FIG. 1. As in FIG. 1, the pack portion of the backpack is not shown to more easily view the exoskeleton. The IV bag is shown to provide orientation of the components of the invention for display purposes.

FIG. 3 shows the front view of the backpack exoskeleton, as embodied in FIG. 1, but without showing a wearer W. Also shown is the ambulatory intravenous socket hanger assembly. The IV bag is shown to provide orientation of the components of the invention for display purposes.

FIG. 4 shows the opposite side view of the backpack exoskeleton of FIG. 2, but without showing a wearer W. Also shown is the ambulatory intravenous socket hanger assembly. The IV bag is shown to provide orientation of the components of the invention for display purposes.

FIGS. 5a and 5b show detailed views of the intravenous socket hanger assembly. FIG. 5a shows a side view of the embodiment. FIG. 5b shows a side view of a telescoping intravenous socket hanger assembly.

FIG. 6 provides a perspective of an alternative embodiment of the invention showing a straight intravenous or IV hanger assembly wherein an intravenous fluid bag (shown for orientation and display purposes) is attached to the backpack exoskeleton by a clamping device.

FIG. 7 shows a detailed view of a clamping device embodiment, focusing on a traditional intravenous hanger assembly.

### DETAILED DESCRIPTION OF EMBODIMENTS OF THE INVENTION

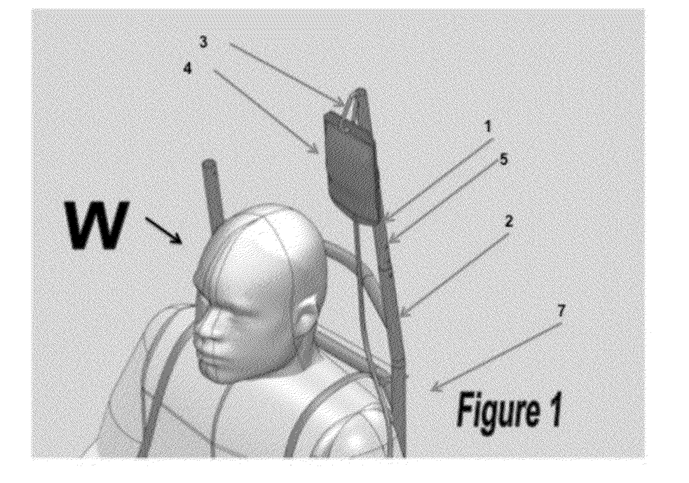

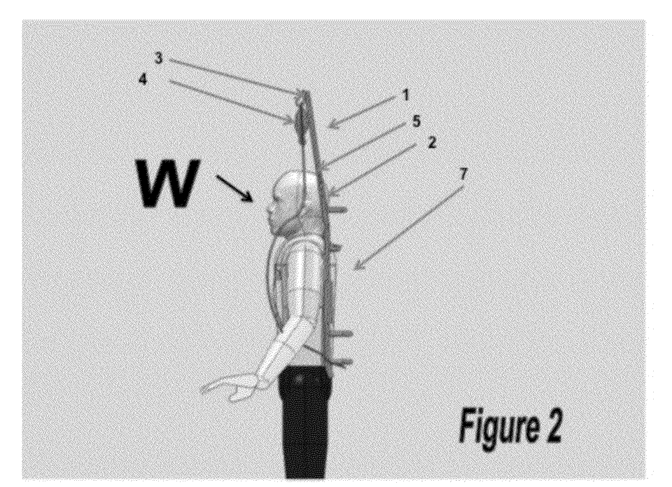

The invention is most easily described by reference to the drawings, but the invention is not limited thereto. Beginning with FIGS. 1 and 2, an exemplary embodiment of the device is depicted as fully configured, but showing only the exoskeleton, minus the backpack, for viewing simplicity. The invention of FIGS. 1 and 2 may be worn or used in either configuration, i.e., with or without the backpack attached to the exoskeleton.

As shown in the Figures, a commercially available exoframe-supported backpack is selected from substantially any framed backpack, including backpacks for hiking or backpacking that have internal frames, backpacks for hiking or backpacking that have external frames, lightweight daypacks and the like. The term “backpack” as used herein is intended to encompass any pack that is worn on the back of a wearer, including any known or yet unknown model of same, since such devices are well known in the art for recreational camping or hiking, purposes, or military purposed, or as adapted for hydration or search and rescue purposes. The term “backpack,” as used herein, is intended to encompass shoulder-borne exoframe supported carriers of known styles, for example, a conventional backpack including shoulder straps and a hip belt for securing to the wearer, and the like. Optionally, pack portion of the backpack includes one or more internal and/or outer pockets, for organizing, separating, or otherwise arranging stored items, closable by zipper or any slidable sealing feature, snap or VELCRO®, are known in the art and are commercially available and can be modified in accordance with the present disclosure. The area of the exoframe that contacts the wearer's back may further be padded to enhance comfort.

As shown, the backpack is configured to be secured to wearer W, and when so secured has generally bilateral symmetry with respect to the spinal column of the wearer W.

FIGS. 1 and 2 both show the invention as it would appear on a wearer W, but of course the wearer is not actually a part of the invention.

The term “wearer W,” as used in connection with the present invention, encompasses any patient in need of an IV infusion, who is wearing the device, specifically a human. But in alternative embodiments, the invention encompasses intended use by a wearer W, who is not the patient, but who is adjacent to the patient and wearing the invention on behalf of the patient needing the IV infusion. The exemplary device is shown in FIGS. 1 and 2 in the adjusted state with the IV fluid pouch attached and configured for use.

As shown in FIGS. 1 and 2, an exemplary embodiment of the present invention provides a straight intravenous socket hanger assembly 1, comprising either construction that slides into exoskeleton 2 of the backpack assembly, or that slides over said exoskeleton 2 (not shown). Moreover, the view of the invention as attached to the exoskeleton, without the pack portion, is shown in FIGS. 1 and 2.

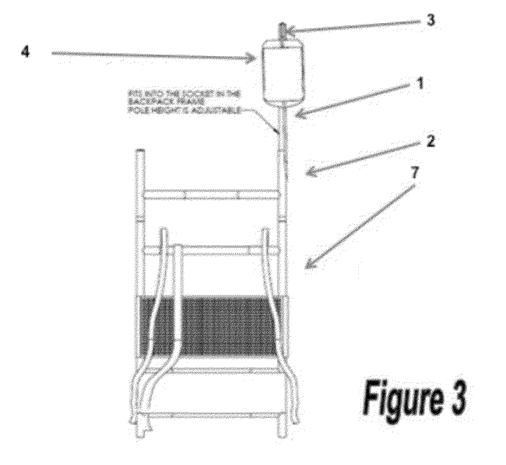

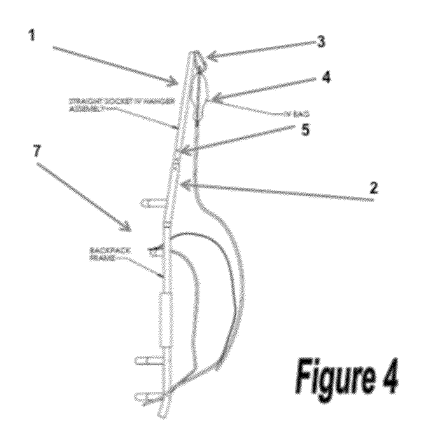

FIGS. 3 and 4 show, respectively a front view of the embodiment of FIG. 1 (without a wearer W) and the side view of the embodiment of FIG. 1. However, the wearer W is not shown in either of these Figures to open the view of the depicted invention. Moreover, the view of the invention is shown, as attached to the exoskeleton without the pack portion, in FIGS. 3 and 4.

The IV bag is shown in FIGS. 1-4 for orientation of the components of the invention for display purposes. When the invention is in operable use, an IV bag is attached to the invention to deliver the IV infusion to the patient in need thereof. However, the IV bag per se is not an element of this invention. Consequently any IV bag by any manufacturer may be used in the practice of using the present invention, with the proviso that the IV bag must be of standard configuration known in the art for such gravity-fed, medical IV bags, and intended for hanging from a hole or point at the center top of the bag. Consequently, the standard IV bag configuration permits the hook or clip 3 of the present invention to be affixed to the bag when it is placed in operation.

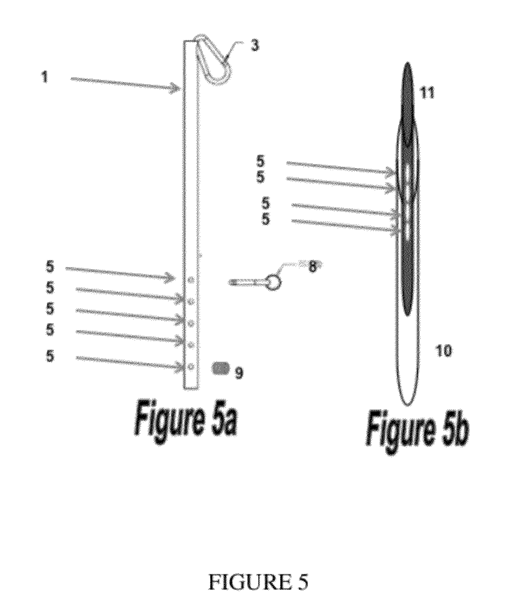

FIG. 5 (FIGS. 5a and 5b) shows the exemplary straight intravenous socket hanger assembly of the embodiment of the present invention shown in FIG. 1. One embodiment of the intravenous socket hanger assembly comprises a singular tubular member or pole segment 1. In the depicted embodiment, the pole segment 1 is approximately 24 inches long. However, the length of IV pole 1 is not so limited, and it may actually range in length from 12 inches to 30 inches in approximately one inch increments. The length of pole 1 is functionally defined in terms of the dimensions of exoframe 2 that supports pole 1, which may be adjusted/tailored to the size and strength of wearer W, or more simply determined by the selection of the wearer.

The material composition of adjustable IV pole 1 in the exemplary embodiment is aluminum, but it need not be so limited to only aluminum. Clearly the adjustable pole is preferably made of a lightweight tubular material, such as aluminum, but other materials known in the art are suitable for such purposes. Such materials include hard plastics, polypropylene, or other durable, inexpensive, and lightweight metals, such as titanium or steel or carbonaceous materials, or even wood. The material is not important so long as it is formable into the size required to fit into the receptacle tube on the exoframe, and it is lightweight and durable, having sufficient rigidity to securely support the combined weight of the apparatus, the IV bag when in use, and the contained medical IV fluid being delivered when the invention is in operable use. The adjustable IV pole is tubular, preferably, but not necessarily a hollow tube.

In an alternative embodiment, shown in FIG. 5b, adjustable pole 1 is configured as a telescoping device, comprising a plurality of tubular members 10, 11, and/or pole segments. “Telescoping” is used herein according to its generally understood definition, meaning to slide inward or outward in, or as if in overlapping cylindrical sections. For example, rather than one long piece, such as the 24 inch member described above, tubular members 10, 11 comprise, for example, two 12½ inch lengths (or other extension lengths, wherein each telescopes within the adjoining member in 2, 3 or 4 or more segments totaling the selected final length of the extended pole), but which, when assembled and fully extended, would measure a total extended length of e.g., in the example of approximately 24 inches, with one inch overlap between each set of segments.

As will be understood by one of ordinary skill in the art, with an understanding of tubular extensions, the number of tubular members and the actual and relative length of the tubular members, described herein, are exemplary and illustrative. They are not a limitation of the invention; variations in the materials used, the number of tubular members and the actual and relative length of each tubular member are possible without departing from the spirit of the present invention as disclosed. To permit telescoping, the extensions must be of decreasing or increasing diameter as compared to the attached tubular member. In a preferred embodiment of the present invention the extensions are of decreasing diameter. Each subsequent telescoping tubular member 10, 11, wherein each interfitting member is respectively sufficiently smaller (in terms of diameter of the respective tubes) to slidably, but securely fit inside the preceding tubular member 10, 11, respectively, as is operationally understood in the art.

The inner diameter of the tubular members of exemplary exoskeleton 2 is preferably approximately 0.5 inch. In alternative embodiments, however, the selected inner diameters, respectively, of the exoskeleton tubular member range from 0.25 in to 1.0 inch at 0.10 inch increments, encompassing tubular members as may be formed or found in a commercially available backpack having exoframe 7. As indicated above, the diameter of the selected tubular members are functionally defined in terms of strength and weight that is light to wear, but sufficiently strong as to support the combined weight of the invention, with the added weight of an IV bag and fluid contents when used in operation. Moreover, if telescoping, then the tubular members must be sized as described above to permit telescoping of the subsequent telescoping member.

The outer diameter of the exemplary tubular members, such as adjustable IV pole 1 is generally approximately 7/16 inch, but the invention encompasses any pole having a diameter that will adequately support IV bag 4 when filled with IV fluid. As above, however, in alternative embodiments, however, the selected outer diameters, respectively, of the exoskeleton tubular member ranges from 0.25 in to 1.0 inch at, e.g., 0.10 inch increments, encompassing tubular members as may be formed or found in a commercially available backpack having an exoframe 7. Moreover, if telescoping, then the tubular members must be sized as described above to permit telescoping of the subsequent telescoping member.

As embodied in FIG. 5, exoskeleton 7 has a series of horizontal thru-holes 5 located at intervals in pole 1. In the exemplary embodiment of exoskeleton 7 shown in FIGS. 5a and 5b there are 4 evenly-spaced horizontal thru holes 5 in the side of the exoskeleton supporting the vertical member support for the IV pole 1. In the alternative, holes are placed in both vertical supports 2 of exoskeleton 7. The placement of thru-holes 5 in one or both vertical supports 2 of exoskeleton 7 allows IV pole 1 to be placed in either the left or right side of the exoskeleton depending on the placement of the IV delivery in the patient.

Exemplary IV pole 1, as shown in FIG. 5a, has a top end and a bottom end as viewed in an up and down position, as it would be seen in the orientation shown in FIG. 1 (‘up’ referring to the head end of the wearer W; ‘down’ referring to the feet end of wearer W when standing, as would be expected in an invention utilizing a gravity feed). Thru-holes 5 are placed every 0.5 inch to 1.0 inch, horizontally starting from the bottom end of the pole to permit locking mechanism 8 to hold the added adjustable pole in place within open vertical pole 2 of the exoskeleton (shown in FIG. 1). For example in the exemplified embodiment of FIG. 5a, there are 4 horizontal thru holes 5 located, e.g., at 1 inch, 1.5 inches, 2 inches and 2.5 inches from the bottom of vertical support 2. The spacing is not so limited, but it is provided to exemplify the intended positioning of the thru-holes. As a result, the spacing could also begin at 1 inch, and then be 5 holes spaced at 0.4 inch apart, at 1.4 inch, 1.8 inch, 2.2 inch and 2.6 inch; or be 4 holes beginning at 1.2 inches, spaced at 0.6 inch apart, at 1.8 inch, 2.6 inch, and 3.2 inch, and the like.

Adjustable pole 1 for holding the IV bag 4 (when the IV bag is in operable use) is held in place within vertical support 2 by locking device 8, which may be as simple as a reversibly locking ring or pin-type device, that passes in either direction, through a set of matched holes 5 in the side of vertical support 2 of frame 7, then through adjustable IV pole 1, and then through the other side of the same thru-hole 5 of vertical support 2 of frame 7. The poles 1 are thus held in place with regard to each other, with locking device 8 through the mated, aligned holes 5 (i.e., the aligned holes are “mateable”). The spacing of the holes is not critical per se. The key element is that selected hole 5 on vertical support 2 of frame 7 must be mated and align with the matching hole on adjustable IV pole 1, to securely lock the poles into place within each other as shown.

In an alternative embodiment, a spring-loaded locking device, preferably a mated spring-loaded reversibly retractable button and hole 9 is provided. In a preferred embodiment of this alternative, the button is provided, facing outward from the outer side (“outside” assuming its normal meaning) at the bottom of the side (wherein the pole has a top and a bottom end as the pole is oriented when worn as shown in FIG. 1) of the of adjustable pole 1 for holding IV infusion bag 4. To be operable, the button on the bottom end of the outside surface of adjustable pole 1 faces toward and is aligned with one or more holes provided at intervals along the vertical length of frame member 7. The one or more holes in vertical pole 7 are sized to reversibly mate to and fit over the spring-loaded button, permitting the spring-loaded locking button to pop into the hole when the two are aligned and mated to hold adjustable IV pole 1 in place within the length of vertical frame member 2. The holes for receiving the button may also be holes 5, shown in the Figures and spaced as described above. The design of such spring-loaded locking devices is known in the art, and their operation is known to one skilled in the art. Thus, a selected hole 5 into which the spring-loaded button 9 mates and locks is aligned with the matching selected hole on frame 7 to determine the total height of the device above the head of wearer W. Alternately, the total height is determined to be at a position wherein the bottom of IV bag 4 (the bag having a top end and a bottom end as viewed in the orientation shown in FIG. 1) is operably located above the shoulder of the wearer W when the invention is worn to provide adequate gravitational flow of the IV fluid contained therein during infusion.

Use of additional spring-loaded buttons 9 are used in certain embodiments to lock the extension poles 10, 11 securely in place when multiple tubular lengths are used as described above to form the total length of adjustable IV pole 1.

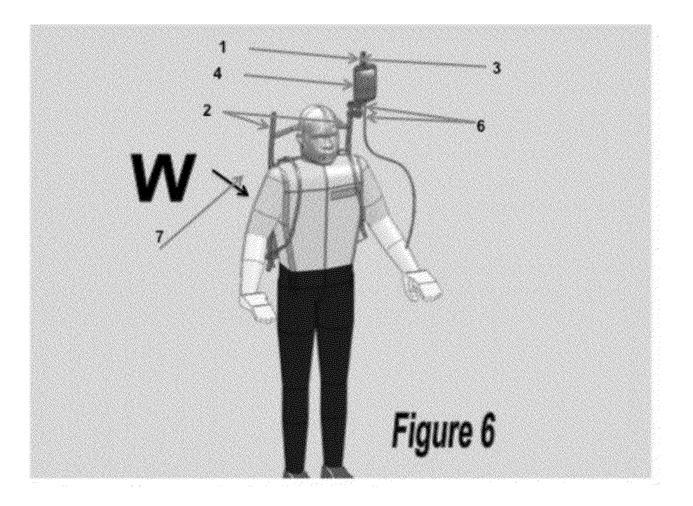

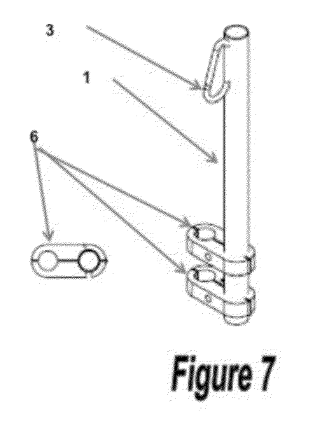

In an alternative design, as shown in FIG. 6, clamping device 6 is attached to vertical frame member 2 of exoskeleton 7. Clamping device 6 is preferably of two part reversibly-mated construction. This clamp, as shown in the detail of FIG. 7, is attached to vertical frame 2 by clamping the two mated halves, each on either side of, and when mated, enclosed around vertical frame member 2, and further comprising a proximate opening for an aligned second tubular member. Alternately clamping device 6 is first attached to adjustable IV pole 1 and then to vertical frame member on the exoskeleton. As a further alternative, both poles are aligned in an offset position next to each other, and then clamped into place by the clamping device. In operation, exemplary IV pole 1 slides into place as shown in FIG. 6 into the proximate opening in the clamping device for the second tubular member, or in the reverse orientation when the clamp encircles the IV pole, the second proximate opening slides over the vertical frame member, and the clamp is drawn tight to reversibly align the two poles. The height of exemplary adjustable IV pole 1 is adjusted by means familiar to one of ordinary skill in the art of adjusting pieces of similar hollow tubular construction held in reversible, but proximate, offset alignment.

Another embodiment of an exemplary extension pole is shown in FIG. 7, providing an exemplary straight intravenous bag hanger 1 in a deployed state. As shown in FIG. 7, clamping device 6 is shown as it is operationally utilized to secure the intravenous hanger assembly to exoskeleton frame 7.

When the ambulatory device or system of the present invention is in operable use forming an IV bag hanger assembly, an IV fluid bag 4 is suspended from the top of adjustable IV pole 1 by reversibly attaching the bag to fluid bag retention device 3, of any type known in the art, including clamps, hooks, snaps, loops, and the like. Such connections are known to one of ordinary skill in the art for attaching two pieces of similar construction together. In certain embodiments, the device or system further includes closures or VELCRO® loops to secure the IV bag in place on the retention device. For example, in one embodiment of the invention, IV fluid bag retention device 3 comprises a locking hook-type device, similar in construction to a carabineer. In operation, IV bag 4 clicks into fluid bag retention device 3 through the typical means of supporting an IV fluid bag, and rests on the bottom of device 3. For example, when using a carbineer type retention device, the opening at the top of the bag (intended to permit hanging the IV bag in standard practice on an IV bag suspension carriage) rests on and hangs from the “J” portion of the closed carabineer.

The exemplary embodiments of the device of the present invention is scalable, and is intended to store and/or support either one or more than one IV bag for simultaneous or sequential use, while still permitting the wearer/patient freedom of movement. Adjusting dimensions, number of members or types of materials still meets the intended requirements and the spirit of the presented exemplary device.

Method for Configuring the Device into Operable Form

The initial configuration of the ambulatory IV transport device 7 is with IV pole 1 in the stowed position, so that the top of pole 1 is flush with the top of vertical support member 2. Top means toward the head end of wearer W when standing in a normal gravity feed orientation of the device.

To assemble the device of the exemplary embodiment, locking device(s) 8 is removed from thru-holes 5 that hold IV pole 1 in place when stored within vertical support member 2.

Adjustable IV pole 1 is then extended by pulling the adjustable IV pole from the top of vertical support 2 until the IV pole reaches the desired height (in terms of operable fit when worn by wearer W). The height of adjustable IV pole 1 is also based on the size of IV bag 4. The bottom of IV bag 4, containing the reservoir of IV fluid, when in operable position as the bottom of the bag is viewed in FIG. 1) preferably is approximately 0.5 inch above the shoulder of wearer W when the wearer is also the patient receiving the infusion. However, in alternative embodiments, the bottom of IV bag 4 can range from between 0 inch to 2 inch above the shoulder, although the 0.5 spacing is presented for simplicity. When the device is worn in the alternative embodiment by an individual adjacent to the patient, the bag must remain above the head of the patient at a level that permits gravitational flow of the IV fluid to the patent.

After extension, adjustable IV pole 1 is then locked into place by placing one or more locking devices 8 back into one or more of a corresponding number of holes 5 in vertical support 2 and adjustable IV pole 1 as described above, at the desired extended length.

Then an IV bag 4 may be attached to the assembled fluid retention device at the end of IV pole 1. The IV system itself is activated by known methods or per manufacturers' instructions.

Once assembled, wearer W can place the ambulatory device comprising exoframe 7 on his/her back in the same manner as putting on any other backpack. In this manner, the wearer W can independently operate the device carrying the IV bag with minimal effort, even if the wearer is a patient in significantly weakened condition, or conversely during vigorous exercise. Finally, the entire system is placed into operation when the output of the IV tubing is attached to the patient or to the input of patient's IV port, thereby permitting IV infusion without limiting the mobility of the patient.

### Methods of Use

The present invention permits an ambulatory device for supporting any infusible fluid (medical or other fluids) into the body of a patient. Although referred to throughout this disclosure as intravenous fluid or IV delivery, the delivery and fluids may also be intended for intra-cavity purposes, and the like.

Advantageously, the delivery methods of the present invention require no pump or electrical connection. The defined hardware permits the medical fluid to be delivered from a bag, pouch, or any container meeting the sterility requirements of safe health practices to an individual by gravity feed, but without attaching the IV bag, pouch, etc, to a pole affixed to a stationary instrument or to a pole on the floor or on wheels on the floor, which would thus also lock the patient into a position of limited mobility during the infusion process. In an alternative embodiment encompassed by this invention, however, the fluid may be delivered by a battery powered pump carried in the backpack. The assembly of the IV delivery system, however, remains the same as for the gravity system.

Accordingly, the herein disclosed invention for the first time gives the patient wearing the assembled device free range of motion, even while the patient is receiving the medical fluid infusions.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. An ambulatory intravenous delivery device, comprising:
at least one tubular adjustable IV pole having a top end and a bottom end;
a fluid bag retention device attached to the top end of the adjustable IV pole;
a wearable shoulder-borne, backpack-type exoskeleton, comprising at least one hollow, tubular, vertical support member mateable to the at least one adjustable IV pole; and
a locking means between the adjustable IV pole in a position mated to the vertical support member.

2. The ambulatory intravenous delivery device of claim 1, wherein
the at least one tubular vertical support member further comprises at least one thru-hole passing horizontally across its diameter;
the adjustable IV pole further comprises at least one horizontal thru-hole passing horizontally across its diameter; and
at least one thru-hole of the at least one tubular vertical member support is mated to at least one thru hole of the adjustable pole.

3. The ambulatory intravenous delivery device of claim 2, wherein the locking means reversibly, but securely, affixes the at least one tubular vertical support member to the mated at least one adjustable pole, when the mated thru-holes in each are aligned.

4. The ambulatory intravenous delivery device of claim 2, further comprising a plurality of thru-holes in the at least one tubular vertical member support mated to a plurality of thru-holes in the at least one adjustable pole, spaced so that when mated, the thru-holes are aligned.

5. The ambulatory intravenous delivery device of claim 2, further comprising a plurality of locking means, each having a length sufficient to extend through an equal number of mated thru-holes in the at least one tubular vertical support member and thru holes in the at least one adjustable pole, when the mated thru-holes are aligned.

6. The ambulatory intravenous delivery device of claim 1, further comprising a pack attached to the wearable backpack-type exoskeleton.

7. The ambulatory intravenous delivery device of claim 1, wherein the at least one tubular vertical support member and the at least one adjustable IV pole are constructed to fit in a telescoping orientation to one another when mated.

8. The ambulatory intravenous delivery device of claim 3, wherein the locking means comprises a length sufficient to extend through the thru-hole in the at least one tubular vertical support member and the mated thru hole in the at least one adjustable IV pole, when the mated thru-holes are aligned.

9. The ambulatory intravenous delivery device of claim 3, wherein the locking means comprises a spring-loaded locking button facing outward from the adjustable pole at a point on its bottom side, reversibly mated to at least one hole in the vertical support member, wherein the button is sized to reversibly, but securely, fit within the hole in the vertical pole.

10. The ambulatory intravenous delivery device of claim 1, further comprising a clamping device of reversibly mated construction, attached to and enclosing the vertical frame member in proximate, offset alignment to the adjustable IV pole on the exoskeleton.

11. An ambulatory intravenous delivery system, comprising:
a wearable, shoulder-borne exoskeleton comprising at least one hollow, tubular, vertical support member;
at least one adjustable IV pole having a top end and a bottom end as positioned in operation for hanging an IV fluid bag from the top end so that the bag remains above a wearer's shoulder, the adjustable IV pole being mateable to the at least one vertical support member;
a fluid bag retention device attached to the top end of the adjustable IV pole; and
a locking means for securing the adjustable IV pole in a position mated to the vertical support member.

12. The ambulatory intravenous delivery system of claim 11, wherein
the at least one tubular vertical support member further comprises at least one thru-hole passing horizonally across its diameter;
the adjustable IV pole further comprises at least one horizontal thru-hole passing horizontally across its diameter; and
at least one thru-hole of the at least one tubular vertical member support is mated to at least one thru hole of the adjustable pole.

13. The ambulatory intravenous delivery system of claim 12, wherein the locking means reversibly, but securely, affixes the at least one tubular vertical support member to the mated at least one adjustable pole, when the mated thru-holes in each are aligned.

14. The ambulatory intravenous delivery system of claim 12, further comprising a plurality of thru-holes in the at least one tubular vertical member support mated to a plurality of thru-holes in the at least one adjustable pole, spaced so that when mated, the thru-holes are aligned.

15. The ambulatory intravenous delivery system of claim 12, further comprising a plurality of locking means, each having a length sufficient to extend through an equal number of mated thru-holes in the at least one tubular vertical support member and thru holes in the at least one adjustable pole, when the mated thru-holes are aligned.

16. The ambulatory intravenous delivery system of claim 11, further comprising a pack attached to the wearable backpack-type exoskeleton.

17. The ambulatory intravenous delivery system of claim 11, wherein the at least one tubular vertical support member and the at least one adjustable IV pole are constructed to fit in a telescoping orientation to one another when mated.

18. The ambulatory intravenous delivery system of claim 13, wherein the locking device comprises a length sufficient to extend through the thru-hole in the at least one tubular vertical support member and the mated thru hole in the at least one adjustable IV pole, when the mated thru-holes are aligned.

19. The ambulatory intravenous delivery system of claim 13, wherein the locking device comprises a spring-loaded locking button facing outward from the adjustable pole at a point on its bottom side, reversibly mated to at least one hole in the vertical support member, wherein the button is sized to reversibly, but securely, fit within the hole in the vertical pole.

20. The ambulatory intravenous delivery system of claim 11, further comprising a clamping device of reversibly mated construction, attached to and enclosing the vertical frame member in proximate, offset alignment to the adjustable IV pole on the exoskeleton

21. A method for using the ambulatory intravenous delivery device of claim 1 for supporting a fluid bag, the method comprising:
extending the adjustable IV pole;
locking or clamping the adjustable pole into a position relative to the at least one tubular vertical member support of the exoskeleton; and
attaching a fluid bag to the IV fluid bag attachment device thereby permitting infusion.

22. A method for using the ambulatory intravenous delivery device of claim 1 to infuse fluid into a patient, the method comprising:
extending the adjustable IV pole;
locking or clamping the adjustable pole into a position relative to the at least one tubular vertical member support;
attaching a fluid bag to the fluid bag attachment device on the extended adjustable IV pole; and
connecting an output of the fluid bag to the patient.
